(19) United States
(12) Patent Application Publication
(10) Pub. No.: US 2007/0073067 A1
(43) Pub. Date: Mar. 29, 2007

(54) Supercritical fluid phase synthesis of methylene lactones using catalysts derived from hydrotalcites

(76) Inventors: Keith W. Hutchenson, Lincoln University, PA (US); Kostantinos Kourtakis, Media, PA (US); Leo Ernest Manzer, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/452,638

(22) Filed: Jun. 14, 2006

Related U.S. Application Data
(60) Provisional application No. 60/711,137, filed on Aug. 25, 2005.

(51) Int. Cl. C07D 307/02

(52) U.S. Cl. 549/295

(57) ABSTRACT

Supercritical fluid phase process for converting certain lactones to their alpha-methylene substituted forms using a catalyst made from a thermally decomposed hydrotalcite.

### CROSS-REFERENCE TO RELATED APPLICATION

This application claims priority under 35 U.S.C. §119 from U.S. Provisional Application Ser. No. 60/711,137, filed Aug. 25, 2005.

### FIELD OF INVENTION

This invention pertains to a method of producing unsubstituted and substituted alpha-methylene lactones by a supercritical fluid phase reaction of starting lactones with formaldehyde in the presence of a catalyst derived from a hydrotalcite.

### BACKGROUND

Alpha-methylene-gamma-butyrolactone and methyl alpha-methylene-gamma-butyrolactone are useful monomers in the preparation of both homopolymers and copolymers. In addition, the alpha-methylene-gamma-butyrolactone group is an important structural feature of many sesquiterpenes of biological importance.

U.S. Pat. No. 6,649,776 and US 2003/0166949 describe a method for converting certain starting lactones to alpha-methylene substituted lactones in a supercritical fluid using a so-called basic catalyst that is made by treating silica with an inorganic salt of K, Rb, Cs, Ca, and Ba. A problem with silica-based catalysts is that they are hydrothermally unstable under reaction conditions involving temperatures above about 200° C. In addition, regeneration cycles involving air produce water at high temperature, and the water can change the porosity and activity of the catalyst

The prior art in this area involves the use of supported catalysts on silica, which are known to be hydrothermally unstable (see for instance, WO9952628A1). Under reaction conditions, or after repeated regeneration cycles, a hydrothermally unstable material will show catalytic performance that will deteriorate with time.

Hydrotalcites are layered, double hydroxides of the general formula
(M²⁺₁₋ₓM³⁺ₓ(OH)₂)(Aⁿ⁻_{x/n}).yH₂O
wherein the M²⁺ ions can be a variety of divalent cations (e.g., Mg, Ni, Zn, Co, Fe, Cu) and the M³⁺ ions can be Al, Fe or Cr. Some hydrotalcites are described by V. K. Diez, C. R. Apesteguia, and J. I. DiCosimo (Latin American Applied Research, 33, 79-86 (2003)) and N. N. Das and S. C. Srivastava (Bull. Mater. Sci. 25, (4), 283-289 (2002)).

Although hydrotalcites are known, and the thermal decomposition of them is known to produce materials that are catalytic for some purposes, their thermal decomposition to produce catalysts for lactone methylenation has not been described. The catalytic activity of thermally decomposed hydrotalcites for lactone conversion reactions cannot be predicted because of the unpredictable nature of catalysis in general.

It would be advantageous, however, to have a lactone methylenation catalyst that is hydrothermally stable at high temperatures and whose activity does not decay appreciably with time on stream (TOS) (after an initial drop in activity) or after several high temperature oxidizing regenerations.

### SUMMARY OF THE INVENTION

This invention is based on the discovery that catalysts derived from hydrotalcites (as described below) are surprisingly active for lactone methylenation, with the advantage that they should possess superior hydrothermal stability compared to prior art supported silica catalysts.

The present invention is a process for preparing a reaction product comprising an alpha-methylene lactone of the Formula II, said process comprising combining a lactone of the Formula I with formaldehyde derived from a formaldehyde source and a solvent to produce a reaction mixture; 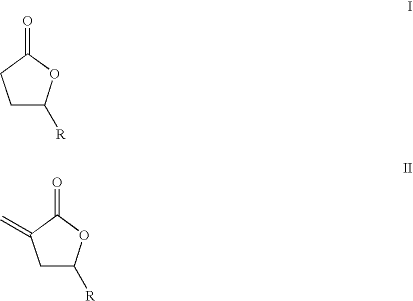
wherein R is selected from the group consisting of hydrogen, methyl, ethyl, and straight or branched C₃-C₅ alkyl;
at a temperature and pressure sufficient to cause the reaction mixture to exist as a supercritical fluid, said temperature being sufficient to cause the formation of said alpha-methylene lactone of Formula II; said reaction mixture being in the presence of a catalyst derived from a hydrotalcite of the formula:
(M²⁺₁₋ₓM³⁺ₓ(OH)₂)(Aⁿ⁻_{x/n}).yH₂O
wherein
M²⁺ is Mg, or a combination of Mg and at least one member selected from the group consisting of Zn, Ni, Co, Fe, and Cu;
M³⁺ is Al, or a combination of Al and at least one member selected from the group consisting of Fe and Cr;
x is 0.66to 0.1; and
A is CO₃ with n=2 or OH with n=1;
by a process comprising heating the hydrotalcite for a time and a temperature sufficient to cause a diminution in the hydrotalcite powder X-ray diffraction pattern peak intensities between 20 angles of 10 degrees and 70 degrees using CuKα radiation.

Up to about one third of the Mg may be substituted with at least one member selected from the group consisting of Zn, Ni, Co Fe and Cu, and up to about one third of the Al may be substituted with at least one member selected from the group consisting of Fe and Cr. In a preferred embodiment of this invention, M²⁺ is Mg, M³⁺ is Al and Aⁿ⁻ is CO₃²⁻. In the process of this invention, the hydrotalcite can be optionally promoted (in the catalytic sense) with at least one group I cation selected from the group consisting of Li, Na, K, Rb, and Cs.

In a highly preferred embodiment, the hydrotalcite is one in which M²⁺ is a Zn and Mg combination, M³⁺ is Al, and x=0.382 with atomic ratios of Zn 0.16/Mg 0.46/Al 0.382. In another preferred embodiment, the catalyst is based on a decomposed hydrotalcite of the formula
(M²⁺₁₋ₓM³⁺ₓ(OH)₂)(Aⁿ⁻_{x/n}).yH₂O,
where M²⁺ is Mg, M³⁺ is Al, x=0.35, promoted with K supplied in the form (Y⁺¹)_{z}(A^{m-}_{z/m}), where z=0.2, and Y⁺¹ is K.

The hydrotalcite-derived catalyst can be made by a process comprising:
(a) combining at least one aluminum salt and at least one magnesium salt, and optionally at least one salt of an element selected from the group consisting of Zn, Ni, Co, Fe, Cu, Cr, Li, Na, K, Rb and Cs to form an aqueous solution;
(b) optionally heating the aqueous solution to 60° C.;
(c) adjusting the pH of the material produced in step (a) or step (b) with base or sodium carbonate to precipitate any hydroxides or hydroxide carbonates that are formed;
(d) drying the material produced in step (c) to produce a hydrotalcite; and
(e) heating the hydrotalcite produced in step (d) for a time and at a temperature sufficient to cause a diminution in the hydrotalcite powder X-ray diffraction pattern peak intensities between 2θ angles of 10 degrees and 70 degrees using CuKα radiation.

### BRIEF DESCRIPTION OF THE DRAWING

The Drawing consists of three figures.

FIG. 1 is a powder X-ray diffraction pattern of a typical hydrotalcite, and

FIG. 2 is a powder X-ray diffraction pattern of the same hydrotalcite after thermal decomposition.

FIG. 3 is a powder X-ray diffraction pattern of a thermally decomposed hydrotalcite referred to herein as “Catalyst 2.” FIG. 3 also shows the peaks of phases resulting from the decomposition: MgAl₂O₄ and MgO.

### DETAILED DESCRIPTION OF THE INVENTION

The following terms generally are abbreviated as follows:
alpha-methylene-gamma-butyrolactone is abbreviated MBL;
gamma-butyrolactone is abbreviated GBL;
gamma-valerolactone is abbreviated GVL;
alpha-methylene-gamma-valerolactone is abbreviated MVL;
gamma-methyl alpha methylene gamma butyrolactone is abbreviated MeMBL;
gas chromatography is abbreviated GC;
mass spectroscopy is abbreviated MS;
time on stream is sometimes abbreviated TOS;
centimeter is abbreviated cm;
degree is abbreviated deg;
mega Pascal is abbreviated MPa;
weight is abbreviated wt;
gram is abbreviated g;
degrees Centigrade is abbreviated ° C.;
milliliter is abbreviated ml;
second is abbreviated sec;
milliampere is abbreviated mA;
kilovolt is abbreviated kV; and
standard cubic centimeters is abbreviated sccm.

The process of the present invention concerns a supercritical fluid phase methylenation of lactones of Formula I to yield alpha-methylene lactones of Formula II.

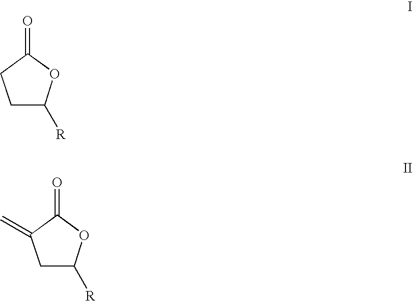

Specifically, lactone of Formula I is reacted with formaldehyde to give a reaction product comprising alpha methylene lactones of Formula II. The substituent —R group is selected from the group consisting of hydrogen, methyl, ethyl, and straight or branched C₃-C₅ alkyl.

In a preferred embodiment, the lactone of Formula I is gamma-butyrolactone (R is H) and the alpha-methylene lactone of Formula II is alpha-methylene-gamma-butyrolactone. In another preferred embodiment, the lactone of Formula I is methyl gamma-butyrolactone (R is methyl) and the alpha-methylene lactone of Formula II is gamma-methyl alpha-methylene gamma-butyrolactone.

The process of the present invention is conducted at reaction conditions to achieve a supercritical fluid state. The temperature is in the range of from about 70° C. to about 400° C. A temperature in the range of from about 100° C. to about 350° C. is preferred. A temperature in the range of from about 200° C. to about 350° C. is most preferred. The pressure will be selected to achieve a supercritical fluid phase at the chosen temperature for a given solvent. Typical pressures for a reaction conducted in a carbon dioxide solvent are in the range of from about 7 MPa to about 60 MPa, with a preferred range of from about 15 MPa to about 40 MPa. The catalyst contact time can be selected to achieve desired yields and selectivities. Contact time can be manipulated by increasing or decreasing flow rates over the catalyst.

The lactones of Formula I, formaldehyde, and solvent are in a homogeneous supercritical fluid phase over the thermally decomposed hydrotalcite catalyst.

The formaldehyde may be supplied to the reaction in the form of an aqueous solution (formalin), anhydrous formaldehyde, formaldehyde hemiacetal, a low molecular weight polyformaldehyde (paraformaldehyde), or formaldehyde trimer (trioxane). The use of paraformaldehyde, trioxane, or anhydrous formaldehyde is preferred since this reduces the need to remove water from the process. Hemiacetals work effectively, but require separate steps to release the formaldehyde from the alcohol and to recover and recycle the alcohol.

The catalyst used in the present invention is made from a hydrotalcite having the formula:
(M²⁺₁₋ₓM³⁺ₓ(OH)₂)(Aⁿ⁻_{x/n}).yH₂O

wherein M²⁺ is Mg, or a combination of Mg and at least one member selected from the group consisting of Zn, Ni, Co, Fe, and Cu; and M³⁺ is Al, or a combination of Al and at least one member selected from the group consisting of Fe and Cr; x is 0.66 to 0.1 and A is CO₃ with n=2 or OH with n=1. In the process of this invention, the hydrotalcite optionally can be promoted with at least one group I cation selected from the group consisting of Li, Na, K, Rb, and Cs, where such group I cations are supplied as oxides, hydroxides, or carbonates according to the formula (Y⁺¹)_{z}(Q^{m-}_{z/m}), where z=0.05 to 0.4, Y⁺¹ is Li, Na, K, Rb or Cs, and Q is O (m=2), CO₃ (m=2), or OH (m=1).

The catalyst can be made by a process (is obtainable by a process) that comprises heating the hydrotalcite for a time and at a temperature sufficient to cause a diminution in the hydrotalcite powder X-ray diffraction pattern peak intensities between 2θ angles of 10 degrees and 70 degrees using CuKα radiation.

More specifically, the hydrotalcite-derived catalyst can be made by a process comprising:
(a) combining at least one aluminum salt and at least one magnesium salt, and optionally at least one salt of an element selected from the group consisting of Zn, Ni, Co, Fe, Cu, Cr, Li, Na, K, Rb, and Cs to form an aqueous solution;
(b) optionally heating the aqueous solution to 60° C.;
(c) adjusting the pH of the material produced in step (a) or step (b) with base or sodium carbonate to precipitate any hydroxides or hydroxide carbonates that are formed;
(d) drying the material produced in step (c) to produce a hydrotalcite; and
(e) heating the hydrotalcite produced in step (d) for a time and at a temperature sufficient to cause a diminution in the hydrotalcite powder X-ray diffraction pattern peak intensities between 2θ angles of 10 degrees and 70 degrees using CuKα radiation.

The salts may be any water-soluble salt including, without limitation nitrates, acetates, acetylacetonates, chlorides, and sulfates.

The starting hydrotalcite materials can be characterized by their powder X-ray diffraction characteristics. For example, powder x-ray diffraction data can be obtained with a PANALYTICAL X'PERT automated powder diffractometer, Model 3040 (Almelo, The Netherlands). Samples are run in a batch mode using a Model PW3065 or PW1775 multi-position sample changer. The diffractometer is equipped with automatic variable slits, a xenon proportional counter, and a graphite monochromator. The radiation can be CuKα (45 kV, 40 mA). Data are typically collected at room temperature from 2 to 90 degrees 2θ; a continuous scan with an equivalent step size of 0.03 deg; and a count time of 2.0 sec. per step. If an alternative radiation is used (e.g. CoKα) the diffraction angles can be recomputed to the radiation of Cu wavelength by using the relation 2d sin θ=nλ, where λ=the wavelength of the X-ray radiation, and θ is ½ of the 2θ value that is typically used in X-ray diffraction patterns.

The diffraction pattern of a hydrotalcite is typically indexed on a rhombohedral or hexagonal unit cell. It is typically a layered structure. Typical diffraction lines have reflections at 11.28 degrees 2θ, 22.78, degrees 2θ, and 34.46 degrees 2θ, which correspond to the crystal composition (Mg₆ Al₂(OH)₁₆)CO₃.4H₂O (Ross, G.; Kodama, H., Am. Mineral., 52 1036 (1967)). This corresponds to x=0.333, n=2, A is CO₃, y=4, M²⁺ is Mg, M³⁺ is Al in the formula:
(M²⁺₁₋ₓM³⁺ₓ(OH)₂)(Aⁿ⁻_{x/n}).yH₂O

Other hydrotalcite compositions exhibit very similar diffraction patterns. However, the position of the peaks will shift slightly depending on the crystallographic unit cell of the other hydrotalcites. Hence, in FIG. 1 (taken from N. N. Das, S. C. Srivastava, Bull. Mater. Sci., Vol 25, no. 4, 283-289 (2002)), an essentially similar pattern will be obtained, but with a slight shift in the 20 positions of the first three intense peaks.

Mg₆ Al₂(OH)₁₈.4.5 H₂O (Mascolo, M. Mineral. Mag., 43 619 (1980), corresponding to x=0.333, n=1, A is OH, y=4.5, M²⁺ is Mg and M³⁺ is Al in the formula:
(M²⁺₁₋ₓM³⁺ₓ(OH)₂)(Aⁿ⁻_{x/n}).yH₂O
shows diffraction peaks at 11.335, 22.841, and 34.742 degrees 2θ, in essentially the same pattern, which characterizes this phase with slightly changed diffraction angles.

Precipitation of the aqueous solution of magnesium or aluminum salts, preferably nitrates, can be accomplished using sodium hydroxide or sodium carbonate. In the former case, if care is given to ensure the absence of contact of the material with CO₂, the pure hydroxide hydrotalcite phase is formed. In the case of reaction with sodium carbonate, a carbonate-containing hydrotalcite phase is formed.

The starting hydrotalcite may be thermally decomposed using conditions (time, temperature and atmosphere) to accomplish the diminution of the intensity of the powder X-ray diffraction peaks characteristic of the hydrotalcite phase.

Heating can be accomplished in air or in a combination of an inert gas such as nitrogen, argon, krypton for parts of the cycle. If a carbonate-free hydrotalcite is desired (A is OH⁻), heating must be accomplished in the absence of CO₂ or CO₂ generating reagents. In that case, an inert atmosphere is desired for all heating steps.

The hydrotalcite can be optionally dried at 120° C. in nitrogen, another inert gas or air (air in the case of carbonate containing hydrotalcites, or A is CO₃²⁻) for a period of 30 minutes to 2 hours. Following the drying step, the hydrotalcite can be heated in air (for A is CO₃²⁻) or nitrogen to a temperature of approximately 350 to 550° C. for a period of approximately 30 minutes to 48 hours. A heating rate of about 5° C./minute is preferred. The exact choice of temperature and heating time at temperature, or the number of these heating cycles, will depend on the hydrotalcite composition and its thermal stability. The conditions needed for any given composition can be chosen based on an examination of the powder X-ray diffraction patterns of the heated materials. The extent of the decomposition of the hydrotalcite can be determined by examining the diminution of the intensities of the first three X-ray diffraction peaks of the hydrotalcite phase as shown in FIG. 1. Typically, greater than 30% reduction in the X-ray diffraction peak intensity means that a portion of the hydrotalcite has decomposed, and this material, which now contains a decomposed hydrotalcite material, is within the scope of this invention.

After the hydrotalcite is thermally decomposed, the intensities of the first three major peaks will be diminished, as shown in FIG. 2 (also taken from Das, et al.).

In some cases, reaction conditions may result in a decrease of catalyst efficiency. In these situations it may be useful to periodically reactivate the catalyst. For example, contacting the present catalysts, when activity drops below an acceptable level, with oxygen at elevated temperatures may have the effect of reactivating the catalyst. Contact temperatures with oxygen may range from about 225° C. to about 500° C., with temperatures of about 250° C. to about 425° C. being preferred.

Thermal and hydrothermal stability are required for the catalyst to withstand one or repeated regeneration cycles without permanently degrading catalyst performance.

Selectivities and yields of product may be influenced by the total contact time with the catalyst. As stated previously, yields and selectivities may be increased by adjusting gas and liquid flow rates.

The present method exploits several advantages of using a supercritical fluid (SCF) as the reaction solvent. SCFs are attractive media for conducting chemical transformations, primarily because the solvent and transport properties of a single solution, including the density, can be varied appreciably and continuously with relatively minor changes in temperature or pressure. The density variation in a SCF also influences the chemical potential of solutes and thus reaction rates and equilibrium constants. Thus, the solvent environment can be optimized for a specific reaction application by tuning the various density-dependent fluid properties. For a discussion of advantages and applications of supercritical fluid media for chemistry and catalysis, see Hutchenson, K. W., “Organic Chemical Reactions and Catalysis in Supercritical Fluid Media,” in Supercritical Fluid Technology in Materials Science and Engineering, Y. -P. Sun (ed.), Marcel Dekker: New York (2002), pp. 87-187.

A fluid is in the SCF state when the system temperature and pressure exceed the corresponding critical point values defined by the critical temperature (T_{C}) and pressure (P_{C}). For pure substances, the critical temperature and pressure are the highest at which vapor and liquid phases can coexist. Above the critical temperature, a liquid does not form for a pure substance, regardless of the applied pressure. Similarly, the critical pressure and critical molar volume are defined at this critical temperature corresponding to the state at which the vapor and liquid phases merge. Similarly, although more complex for multicomponent mixtures, the mixture critical state is identified as the condition at which the properties of coexisting vapor and liquid phases become indistinguishable. For a discussion of supercritical fluids, see Kirk-Othmer Encycl. of Chem. Technology, 4^{th} Ed., Vol. 23, pg. 452-477.

In addition to typical factors such as chemical inertness, cost, toxicity, etc., the critical temperature must be considered when selecting a potential solvent for conducting chemical transformations in the SCF regime. For practical applications, thermal and catalytic chemical reactions can only be conducted in a relatively narrow temperature range. Lower temperatures result in unacceptable reaction rates, and higher temperatures can result in significant selectivity and yield losses, as well as catalyst deactivation. To obtain practical solvent densities and the corresponding density-dependent properties, this temperature optimization must be balanced against a general desire to operate in the vicinity of the mixture critical point of the reaction system to fully exploit the potential advantages afforded by SCF operation. The phase behavior of the reaction mixture, which is strongly influenced by the solvent critical temperature, is fundamentally important in defining this operating window, so one should select a solvent to provide the desired phase behavior. The phase behavior of SCF systems can also be manipulated to control the number and composition of coexisting phases, thus controlling both reaction effects, as well as the separation of products from the reaction mixture.

One can visually observe the phase behavior of the reaction mixture by conducting the reaction in a vessel equipped with a transparent window, or by simulating the reaction mixture with a solution of similar concentration in such a vessel. Systematic determination of the phase boundaries of the reaction mixture can be determined by standard techniques using such a vessel that is also equipped with a means of varying the vessel volume at fixed composition and temperature. The vessel is loaded with the various components at the specified composition of the reaction mixture, heated to the reaction temperature, then the solution pressure is varied by changing the vessel volume until a phase transition is visually observed. After measuring the phase boundary of a solution of interest over the range of anticipated compositions, one can define the operating conditions necessary to achieve the supercritical state for conducting the desired reaction.

Any suitable SCF solvent may be used in the process of this invention, including, but not limited to, carbon dioxide, nitrous oxide, sulfur hexafluoride, fluoromethane, trifluoromethane, tetrafluoromethane, ethane, ethylene, propane, propanol, isopropanol, propylene, butane, butanol, isobutane, isobutene, pentane, hexane, cyclohexane, benzene, toluene, o-xylene, water, and mixtures thereof, provided that it is inert to all reagents and products. Preferred SCF solvents include carbon dioxide or a C₁-C₆ alkane, optionally substituted with Cl, F or Br. More preferred is where the supercritical fluids are carbon dioxide, trifluoromethane, pentane, or propane.

Separation and/or purification of the desired products, including MBL or MeMBL, from unreacted starting lactone and/or reaction byproducts may be performed by processes known in the art. A particularly suitable method to recover the desired product is to polymerize MBL in GBL solution, or MeMBL in GVL solution, using standard free-radical polymerization, isolate the polymer by precipitation, and then thermally depolymerize back to MBL or MeMBL, as the case may be, by heating under vacuum. Alternatively, MBL can be separated from GBL by melt crystallization. Another effective method is liquid-liquid extraction.

Non-limiting reactors suitable for the process of the instant invention include a tubular reactor, fluidized bed reactor, fixed bed reactor, and transport bed reactor. The process can be run in either batch or continuous mode as described, for example, in H. Scott Fogler, Elements of Chemical Reaction Engineering, 2^{nd} Edition, Prentice-Hall Inc, CA, 1992.

The reaction can be carried out by passing solutions of the formaldehyde and lactone over the catalyst at elevated temperatures and pressures sufficient to cause the reaction mixture to exist as a supercritical fluid phase.

### EXAMPLES

Catalyst 1:

Decomposed hydrotalcite of the formula
(M²⁺₁₋ₓM³⁺ₓ(OH)₂)(Aⁿ⁻_{x/n}).yH₂O,
where M²⁺ is Mg, M³⁺ is Al, x=0.25

In a one liter round bottom flask, 51.28 g of magnesium nitrate hexahydrate, Mg(NO₃)₂.6H₂O (EM Sciences) and 25.01 g of aluminum nitrate (EM Sciences) were dissolved in approximately 500 ml of water. The solution was heated to 60 to 70° C. Approximately 140 ml of 30 wt % ammonium hydroxide was slowly added to the stirred solution over a period of about 1 hour. After stirring for another 30 minutes at 60° C., the mixture was allowed to cool to room temperature.

The material, which formed cloudy precipitate, was dried overnight at room temperature, in flowing nitrogen, before heating.

The dried material was loaded into an alumina boat and heated in a horizontal tube furnace. The air flow rate corresponded to a linear velocity of 15.6 cm/minute. The material was heated at a rate of 5° C./minute to 120° C.; this temperature (120° C.) was maintained for four hours. It was subsequently heated at a rate of 5° C./minute to approximately 450° C. and then allowed to cool to room temperature in flowing air.

Catalyst 2

Decomposed hydrotalcite of the formula
(M²⁺₁₋ₓM³⁺ₓ(OH)₂)(Aⁿ⁻_{x/n}).yH₂O,
where M²⁺ is Mg, M³⁺ is Al, x=0.35, promoted with K, supplied as (Y⁺¹)_{z}(Q^{m-}_{z/m}), where z=0.2, and Y⁺¹ is K.
Decomposed, ⅛″ Syntal K Hydrotalcite extrudates were obtained from Sud Chemie Corporation (Louisville, Ky.). Powder X-ray diffraction data indicate the crystalline phases are MgAl₂O₄ and MgO, indicating a, decomposed hydrotalcite. This data is shown in FIG. 3.

The reactions were conducted in a continuous fixed bed reactor consisting of a 0.25-inch (0.63-cm) o.d.×0.049-inch (0.12-cm) wall×4.5-inch (11.4-cm) long 316 stainless steel tube packed with the catalyst with a bed depth of 3.0 inches (7.62 cm). The reactor was heated by cartridge-type electrical heaters mounted in an aluminum block enclosing the reactor. The lactone was combined with ethanol hemiacetal as the formaldehyde precursor and metered to the reactor as a liquid feed with a syringe pump. The ethanol hemiacetal was prepared by refluxing a 50 mol % paraformaldehyde solution in ethanol for four hours at 95° C., followed by cooling to room temperature and filtration. The carbon dioxide solvent was metered as a condensed liquid with a second positive-displacement pump, and the two streams were combined and heated prior to entering the reactor. This solution formed a supercritical fluid phase at the reaction conditions. Liquid-phase reactor effluent samples were collected downstream in an ice bath after venting the carbon dioxide, and reaction products were quantified by gas chromatography using diphenyl ether as an internal standard. The reactor pressure was controlled by a backpressure regulator located downstream of the reactor.

The reactant feed solution consisted of 52.3 wt % GVL with the balance made up with the ethanol hemiacetal solution. This solution resulted in a 1.2:1 ratio of formaldehyde to GVL in the reactor feed, which was metered at a rate to provide a specified weight hour space velocity (WHSV) in the reactor. The carbon dioxide flow rate was metered independently to give a final total organic concentration of 5.0 mol % in the reactor feed. The reactor was operated at a temperature of 245° C. and a pressure of about 23.5 MPa. Additional reaction conditions and the corresponding reaction profile showing conversion of GVL to MeMBL are summarized in Table 1.

The data in Table 1 show that reactions done in accordance with the process of the present invention yield the desired products with modest conversion. Catalyst 1 demonstrates modest selectivity to the desired MeMBL product, and Catalyst 2 demonstrates high selectivity. (In the table, g=grams, cat. h=catalyst times hours, and h=hours.)

TABLE 1
Reaction Data

| | Catalyst | WHSV | | GVL | MeMBL |
|---|---|---|---|---|---|
| | Loading | (g GVL/g | TOS | Conversion | Selectivity |
| Catalyst | (g) | cat · h) | (h) | (%) | (%) |
| Catalyst 1 | 1.52 | 1.02 | 0.75 | 3.0 | 64.4 |
| | | | 1.00 | 1.5 | 28.0 |
| | | | 1.75 | 0.6 | 32.6 |
| | | | 2.55 | 0.5 | 37.5 |
| | | | 4.33 | 0.4 | 42.6 |
| Catalyst 2 | 0.67 | 0.70 | 1.08 | 21.6 | 87.0 |
| | | | 1.95 | 8.3 | 93.6 |
| | | | 2.75 | 5.3 | 95.9 |
| | | | 3.58 | 3.7 | 91.6 |

What is claimed is:

1. A process for preparing a reaction product comprising an alpha-methylene lactone of the Formula II, said process comprising combining a lactone of the Formula I with formaldehyde derived from a formaldehyde source and a solvent to produce a reaction mixture; 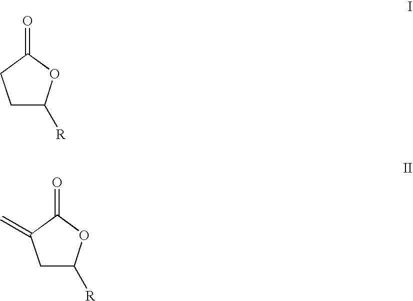
wherein R is selected from the group consisting of hydrogen, methyl, ethyl, and straight or branched C₃-C₅ alkyl;
at a temperature and pressure sufficient to cause the reaction mixture to exist as a supercritical fluid phase, said temperature being sufficient to cause the formation of said alpha-methylene lactone of Formula II; said reaction mixture being in the presence of a catalyst derived from a hydrotalcite of the formula:
(M²⁺₁₋ₓM³⁺ₓ(OH)₂)(Aⁿ⁻_{x/n}).yH₂O
wherein
M²⁺ is Mg, or a combination of Mg and at least one member selected from the group consisting of Zn, Ni, Co, Fe, and Cu;
M³⁺ is Al, or a combination of Al and at least one member selected from the group consisting of Fe and Cr; x is 0.66 to 0.1 and A is CO₃ with n=2, or OH with n=1;
the hydrotalcite optionally promoted with at least one group I cation selected from the group consisting of Li, Na, K, Rb, and Cs by a process comprising heating the hydrotalcite for a time and at a temperature sufficient to cause a diminution in the hydrotalcite powder X-ray diffraction pattern peak intensities between 2θ angles of 10 degrees and 70 degrees using CuKα radiation.

2. The process of claim 1, wherein the catalyst is made by a process comprising:
(a) combining at least one aluminum salt and at least one magnesium salt, and optionally at least one salt of an element selected from the group consisting of Zn, Ni, Co, Fe, Cu, Cr, Li, Na, K, Rb and Cs, to form an aqueous solution;
(b) optionally heating the aqueous solution to 60° C.;
(c) adjusting the pH of the material produced in step (a) or step (b) with base or sodium carbonate to precipitate any hydroxides or hydroxide carbonates that are formed;
(d) drying the material produced in step (c) to produce a hydrotalcite; and
(e) heating the hydrotalcite produced in step (d) for a time and at a temperature sufficient to cause a diminution in the hydrotalcite powder X-ray diffraction pattern peak intensities between 2θ angles of 10 degrees and 70 degrees using CuKα radiation.
